(19) United States
(12) Patent Application Publication
(10) Pub. No.: US 2026/0182930 A1
(43) Pub. Date: Jul. 2, 2026

(54) APPARATUS FOR NON-INVASIVELY COMPUTING CARDIO-VASCULATURE PARAMETERS USING MORPHOLOGY OF UNCALIBRATED PRESSURE WAVE SIGNAL

(71) Applicant: ÉCOLE POLYTECHNIQUE FÉDÉRALE DE LAUSANNE (EPFL), Lausanne (CH)

(72) Inventors: Vasiliki BIKIA, Lausanne (CH); Georgios ROVAS, Lausanne (CH); Nikolaos STERGIOPULOS, Preverenges (CH)

(73) Assignee: ÉCOLE POLYTECHNIQUE FÉDÉRALE DE LAUSANNE (EPFL), Lausanne (CH)

(21) Appl. No.: 19/131,918

(22) PCT Filed: Nov. 17, 2023
(86) PCT No.: PCT/IB2023/061658

§ 371 Date: May 21, 2025

Related U.S. Application Data
(60) Provisional application No. 63/384,852, filed on Nov. 23, 2022.

(51) Int. Cl. A61B 5/00; A61B 5/0205; A61B 5/026; A61B 5/11

(52) U.S. Cl. CPC A61B 5/7278; A61B 5/0205; A61B 5/681; A61B 5/7221; A61B 5/7267; A61B 5/026; A61B 5/11; A61B 2562/066; A61B 2562/164

(57) ABSTRACT

Systems and apparatus are provided for using a non-invasively monitored uncalibrated pressure signal from a patient to compute cardiac output and other cardio-vascular parameters, thereby providing safe, effective, reliable and low cost monitoring of patient health. Wearable devices are disclosed that include a sensor array for obtaining patient data that is used to provide an estimate of aortic systolic blood pressure, cardiac output, and end-systolic elastance that may be determined without the need for a cuff.

### CROSS-REFERENCE TO RELATED APPLICATIONS

The application claims priority to U.S. Provisional Patent Application No. 63/384,852, filed Nov. 23, 2022, the entire contents of which are incorporated herein by reference.

### FIELD OF THE INVENTION

The present invention relates to systems and apparatus for using a non-invasively monitored uncalibrated pressure signal from a patient to compute cardiac output and other cardio-vascular parameters, thereby providing safe, effective, reliable and low cost monitoring of patient health.

### BACKGROUND

A patient's health may be monitored using a number of biomarkers, including the aortic systolic blood pressure, cardiac output, and end-systolic elastance. The significance of these three biomarkers, which previously required invasive measurement, are addressed in turn.

Blood pressure measurements taken using a cuff and sphygmomanometer at the brachial artery has been accepted as an important predictor of future cardiovascular risk. However, systolic pressure varies throughout the arterial tree, such that aortic systolic blood pressure (aSBP) may be lower than corresponding brachial values, although this difference is highly variable between individuals. Emerging evidence suggests a stronger relationship between future cardiovascular events and aortic pressure than brachial pressure. Moreover, anti-hypertensive drugs can exert differential effects on brachial and aortic pressure. Therefore, it would be desirable to base treatment decisions on aortic, rather than brachial, pressure, such as when diagnosing and managing hypertension.

Cardiac output (CO) is a primary determinant of global oxygen transport from the heart to the human body. Cardiac output is considered by some to be a powerful index for predicting clinical outcomes and effectively assessing cardiovascular disease. Critically ill or intensive care unit (ICU) patients often require continuous assessment of CO for diagnostic purposes or for guiding therapeutic interventions. But despite the diagnostic importance of CO, the convenience of its measurement is significantly limited due to the invasive nature of the existing techniques, associated co-morbidities, potential cost, and the need of special equipment or training. Likewise, as a result of these limitations, state-of-the-art methods for obtaining CO are recognized for trend-monitoring rather than for measuring absolute values of CO.

Regarding end-systolic elastance, the clinical need for effectively monitoring cardiac performance, and thus detecting possible myocardial disorders, is well established. However, accurate assessment of the myocardial inotropic state, independently from preload and afterload, remains a challenge. As a result, research over the past decades has been aimed toward deriving a reliable and easily obtainable cardiac index, which offers significant diagnostic value by being a determinant of myocardial contractility and which enables comparison between different pathophysiological states or different individuals by being insensitive to loading conditions. End-systolic elastance (Eₑₛ), i.e. the slope of the end-systolic pressure/volume relation, is a notable determinant of left ventricular (LV) systolic performance and heart interaction with the systemic vasculature. The clinical applicability of the method remains severely limited by two factors: the need for inducing in vivo acute load alterations and the method's invasive nature. It would be desirable to determine Eₑₛ using non-invasive means and without a need for inducing in vivo acute load alterations.

International Application Publication No. WO 2021/033097, which is incorporated herein by reference, describes methods and apparatus for using non-invasively measured physiologic data, such as blood pressure and pulse wave propagation information, to predict in real-time noninvasively unobservable cardiovascular parameters, such as cardiac output, central systolic blood pressure and others. Such values currently are determinable only by way of invasive measurements. In that published application, it is described that a one-dimensional arterial tree may be trained on a limited dataset for a representative patient population, and that trained model then used to generate a database of synthetic data for a larger virtual patient population using an artificial intelligence module. The resulting database for the expanded virtual patient population then may be used to determine key cardiovascular parameters in real-time based only a limited set of non-invasively measured patient data.

Although the methods and apparatus described WO 2021/033097 provide a quick and cost-effective system to obtain estimates of critical cardio-vascular information without invasive measurement, that system still required noninvasive measurement of multiple physiologic parameters, as well as ECG signals and the acoustic detection of heart sounds. Nonetheless, the systems and methods described therein demonstrated the feasibility of using non-invasively measured data to provide accurate real-time estimates of cardiovascular parameters critical to monitoring and assessing patient health.

Other systems and apparatus have been directed to determining cardiovascular functions using sensors in conjunction with a blood pressure cuff. Such devices rely on the use of the blood pressure cuff for calibration of the sensors, and their size and bulkiness are not conducive for portability purposes.

Still other systems and apparatus for determining cardiovascular functions using sensors have been proposed for use by trained medical professionals who have the required knowledge of where to place the sensor to properly obtain accurate input. When operated by non-trained individuals, however, such systems and apparatus lack accuracy and provide unreliable results depending on where the sensors are placed.

Ibrahim et al., Cuffless blood pressure monitoring from a wristband with calibration-free algorithms for sensing location based on bio-impedance sensor array and autoencoder, Scientific Reports, (2022) 12:319, https://doi.org/10.1038/s41598-021-03612-1, describes a cuffless blood pressure (BP) monitoring method based on a bio-impedance (Bio-Z) sensor array built in a flexible wristband that provides a blood pulsatile sensing and BP estimation without calibration methods for the sensing location.

In other systems and apparatus, arterial tonometry is used as a technique that permits noninvasive monitoring of pressure within a superficial artery. It has been found, however, that the pressure determinations from such devices are not easily reproducible and have high variability.

In view of the foregoing, there exists a need for systems and apparatus for estimating key cardiovascular parameters using a simplified set of non-invasively monitored physiologic inputs.

It would be desirable to provide systems and apparatus for predicting key cardiovascular parameters in real time using a limited set of non-invasively monitored physiologic inputs that employs robust software configured to be run without requiring extensive computing resources.

It further would be desirable to provide systems and apparatus for predicting cardiovascular parameters that do not require the use of a blood pressure cuff for calibration.

It still further would be desirable to provide systems and apparatus for predicting cardiovascular parameters that eliminate the need for precise placement of the sensor.

It yet further would be desirable to provide systems and apparatus for predicting cardiovascular parameters that diminish intra- and inter-user variability.

### SUMMARY OF THE INVENTION

The present invention is directed to systems and apparatus for estimating key cardiovascular parameters using a simplified set of non-invasively monitored physiologic inputs. In accordance with one aspect of the invention, portable apparatus is provided that is suitable for reliably and accurately predicting one or more cardiovascular parameters, such as aortic systolic blood pressure, cardiac output, and end-systolic elastance. Advantageously, the inventive apparatus and methods employ an array of sensors that do not require a cuff for calibration, and further form part of a wearable. Moreover, an array of sensors provided in accordance with methods and apparatus disclosed herein may be used by non-trained personnel without issues of increased variability associated with placement for use.

In a preferred embodiment of the present invention, a system for non-invasively monitoring cardiac health of a patient is provided that includes an array of sensors, a receiver, and a controller. The array of sensors is mounted to at least one diaphragm and configured to sense an arterial blood flow, wherein the array includes a first sensor that provides a first signal and a second sensor that provides a second signal. The receiver is configured to receive the first signal and the second signal, and also is configured to analyze a quality of each of the first and second signals and to determine an output signal. The controller is operatively coupled to the receiver and has a processor and non-volatile storage for storing programmed instructions. The instructions, when executed by the controller, generate an estimate for aortic systolic blood pressure, cardiac output, or end-systolic elastance based on the output signal.

The controller may generate the estimate employing a machine learning algorithm, while the array of sensors is mounted on a flexible substrate. The receiver may determine the output signal by comparing the quality of the first signal to the quality of the second signal to select one of the first signal and the second signal as the output signal. Alternatively or in addition, the receiver may determine the output signal by combining the first signal and the second signal. The first sensor may be a pressure sensor, a force sensor, a displacement sensor, an acceleration sensor, a photoplethysmographic sensor, a light reflection sensor, or a light sensor, and at least a plurality of the sensors in the array of sensors are mounted on a common diaphragm. The receiver may be configured to receive the first signal at a different time than the second signal. The array of sensors may be configured to circumscribe a portion of an arm of the patient and additionally the array of sensors may include a motion sensor.

In another preferred embodiment of the invention, a system for non-invasively monitoring cardiac health of a patient is provided that includes a wearable device, a receiver, and a controller. The wearable device includes a flexible substrate and an array of sensors coupled to the flexible substrate. The array of sensors is configured to sense an arterial blood flow, and includes a first sensor of the array that provided a first signal, and a second sensor of the array that provided a second signal. The receiver is configured to receive the first signal and the second signal, and to analyze a quality of each of the first signal and the second signal and to determine an output signal. The controller is operatively coupled to the receiver, and includes a processor and non-volatile storage for storing programmed instructions. The instructions, when executed by the controller, generate an estimate of aortic systolic blood pressure, cardiac output, or end-systolic elastance based on the output signal.

The controller may generate the estimate employing a machine learning algorithm and the receiver may determine the output signal by comparing the quality of the first signal to the quality of the second signal to select one of the first signal and the second signal as the output signal. The receiver may determine the output signal by combining the first signal and the second signal. The first sensor, or second sensor, may be a pressure sensor, a force sensor, a displacement sensor, an acceleration sensor, a photoplethysmographic sensor, a light reflection sensor, or a light sensor.

In yet another embodiment, a system for non-invasively monitoring cardiac health of a patient is provided that includes a wearable device, a receiver, a controller, and a display device. The wearable device includes a flexible substrate on which a first sensor and second sensor are mounted. The first sensor is configured to sense an arterial blood flow and provide a first signal, and the second sensor is configured to sense the arterial blood flow and provide a second signal. The receiver is configured to receive the first signal and the second signal, and is further configured to analyze a quality of each of the first signal and the second signal to determine an output signal. The controller is operatively coupled to the receiver, and has a processor and non-volatile storage for storing programmed instructions. The instructions, when executed by the controller, generate an estimate of aortic systolic blood pressure, cardiac output, or end-systolic elastance based on the output signal. The display device is in communication with the controller, and is configured to display a representation of the estimate.

The controller may generate the estimate employing a machine learning algorithm. The receiver may determine the output signal by combining the first signal and the second signal or the receiver may determine the output signal by comparing the quality of the first signal to the quality of the second signal to select one of the first signal and the second signal as the output signal. The first sensor, or the second sensor, may be a pressure sensor, a force sensor, a displacement sensor, an acceleration sensor, a photoplethysmographic sensor, a light reflection sensor, or a light sensor.

Other features of the inventive system and methods will be apparent with reference to the following description and figures.

### BRIEF DESCRIPTION OF THE DRAWINGS

FIG. 1 is a block diagram identifying functional components of embodiments of the present invention.

FIGS. 2A, 2B, and 2C are cross sections of embodiments of sensor arrays having varied configurations.

FIGS. 3A and 3B are, respectively, configurations of diaphragms depicting an individual diaphragm on one side of a sensor and a diaphragm that surrounds a sensor.

FIG. 4 is an example of a possible configuration of sensors in a sensor array.

FIG. 5 is a representation of an embodiment of a wearable embodiment of the present invention.

FIG. 6 is a functional representation of an embodiment of a sensor array and a receiver.

FIG. 7A is a block diagram of a system constructed in accordance with the present invention, while FIG. 7B is a schematic representation of a machine learning pipeline of an exemplary embodiment of a system constructed in accordance with the present invention.

FIG. 8 is a schematic diagram of a system configured in accordance with the principles of the invention.

### DETAILED DESCRIPTION OF THE INVENTION

The present invention is directed to systems and apparatus for estimating aortic systolic blood pressure (aSBP), cardiac output (“CO”), and end-systolic elastance (Eₑₛ) in real time from uncalibrated non-invasively measured physiologic inputs of a patient. As set forth in the commonly assigned PCT Patent Application No. PCT/IB2023/057976, entitled “Method and Apparatus for Non-Invasively Computing Cardio-Vasculature Parameters Using Morphology of Uncorrelated Pressure Wave Signal,” which is incorporated by reference in its entirety, systems and methods are provided to compute key cardiovascular parameters using simple formulas that relate those parameters to measurements of brachial systolic and diastolic blood pressure, heart rate (HR), and/or pulse wave velocity (PWV) data. This application describes systems and apparatus for obtaining the input measurements and determining the desired parameters.

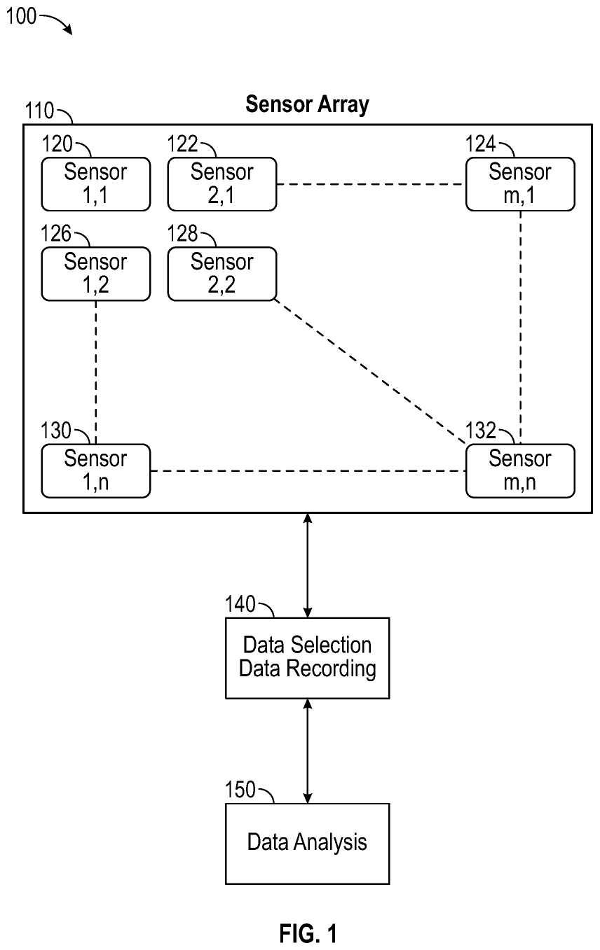

Referring to FIG. 1, some functional components of systems and apparatus in accordance with the present invention are described. System 100 includes sensor array 110 that includes a plurality of sensors distributed in an m by n grid pattern, including illustratively sensor 120, sensor 122, sensor 124, sensor 126, sensor 128, sensor 130 and sensor 132. Sensors 120-132 are selected to detect pulsation data and generate a pulsatile waveform for an arterial location of interest. Users of system 100 may obtain the pulsation data and waveform from a brachial, radial, carotid, temporal or other artery preferably at a depth of 10 cm or less from sensor array 110.

As explained in greater detail below, sensors used in embodiments of the present invention need not be disposed in an m by n grid, and may be arranged in a number of other configurations. Likewise, sensors in a sensor array may, but need not, be of the same type. In some embodiments, it is desirable for all sensors to be of the same type, whereas in other embodiments, it may be desirable to have different types of sensors, such as two or more of a first type of sensor and two or more of a second type of sensor.

Data selection and data recording component 140 is in communication with sensor array 110 to receive signals from sensors in sensor array 110, analyze the signals, and determine an output signal. Data analysis component 150 is in communication with data selection and data recording component 140 to receive the output signal for processing to estimate the desired cardiovascular parameter(s). In some embodiments, data selection and data recording component 140 may be integral with data analysis component 150.

Other functional components may be added to this system, such as a data storage component, for storing one or more of the signals form the sensors, the output signal, or the estimate of the desired cardiovascular parameter(s). Likewise, a data display component may be included for providing visual indicia of one or more of the signals from the sensors, the output signal, or the estimate of the desired cardiovascular parameter(s) to a clinician, patient, or other individual. Sensor array 110, data selection and data recording component 140, and data analysis component 150 may be integrated into a single device or one or more of these components may be located at a distance from the other component(s) and configured to communicate via wires or wirelessly. In some embodiments, signals or other data communicated between the functional components may be compressed to reduce the size of the exchanged data. Likewise, signals or other data being communicated between the functional components may be encrypted for security purposes. Wireless communication between functional components may be provided using any suitable standard, such as WiFi, Bluetooth, or other known communication methods.

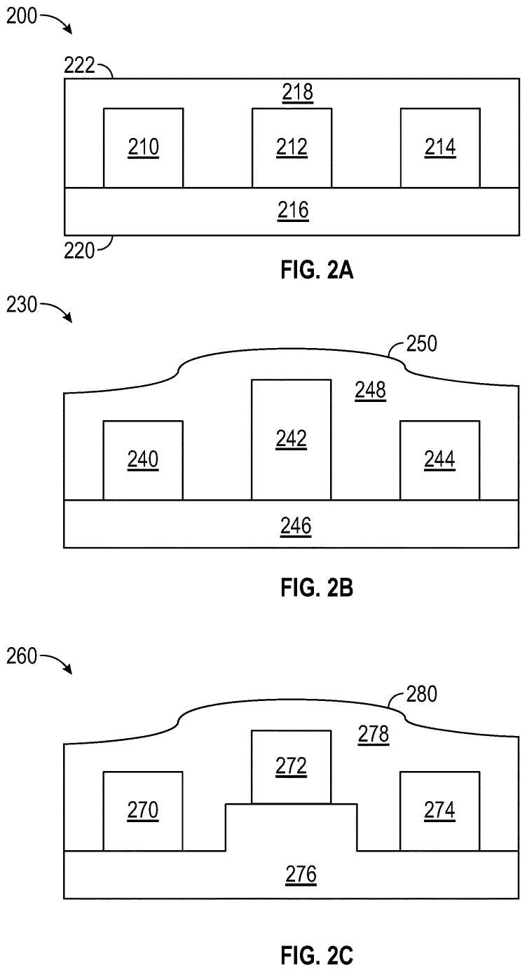

Referring now to FIG. 2A, a portion of sensor array 200 is described. Sensor array 200 includes sensor 210, sensor 212, and sensor 214, all mounted on substrate 216. It will be appreciated that sensor array 200 may include other sensors, which are not shown, for purposes of simplification. Sensors 210, 212, and 214 also are coupled to diaphragm 218. Sensors 210, 212, and 214, like other sensors that may be used with the present invention, may include one or more of pressure, force, displacement, acceleration, photoplethysmographic, light reflection, light transmission, light intensity, ballistocardiographic, ultrasonic, or other type of sensor suitable for sensing pulsation of blood in a targeted artery. Each of sensors 210, 212, and 214 may, but need not, be the same type of sensor.

Sensors 210, 212, and 214 preferably are mounted to substrate 216, which may be rigid, flexible, or semi-flexible. For some embodiments a rigid substrate may be preferred, including embodiments designed to compress and/or manipulate the area of application. In other embodiments, a flexible substrate may be preferred, so as to allow the sensor array to conform to the patient's body contours.

Sensor array 200 also includes diaphragm 218, which in use is disposed between the patient and sensors 210, 212, and 214. In preferred embodiments, diaphragm 218 is a flexible membrane or film that covers all or part of the sensors, which may be wired or wireless. In embodiments in which the sensors are wired, the wires may be disposed within the substrate or alternatively may be disposed within the diaphragm. In wired embodiments lacking a diaphragm, the wires preferably are disposed in or on the substrate, while in embodiments lacking a substrate, the wires preferably are disposed in or on the diaphragm. In still other embodiments, the wires may be exposed or disposed in other material near the sensor array.

Diaphragm 218 may provide a number of advantages. For example, the diaphragm may protect the sensors and other parts from damage caused by contact with external objects. It also may electrically isolate the circuit from the patient. Still further, the diaphragm may provide a coupling between the skin and the sensor along a smooth uniform contact area. In some embodiments with a wearable device, diaphragm 218 may be flexible and designed to look like a watch wristband or bracelet.

While FIG. 2A depicts outer surface 220 of substrate 216 and outer surface 222 of diaphragm 218 as flat surfaces, one of skill in the art will understand that if substrate 216 and diaphragm 218 are each formed of flexible material, then sensor array 200 could be deformed such that outer surface 220 is convex and outer surface 222 is concave. In this manner, sensor array 200 can conform to a curved portion of a patient's body, such as a wrist, leg, or neck.

In some embodiments, it may be desirable to configure the sensor array such that the diaphragm does not have a uniform outer surface, as shown, for example, in FIGS. 2B and 2C.

More specifically, FIG. 2B illustrates sensor array 230 that includes sensor 240, sensor 242, and sensor 244 mounted between substrate 246 and diaphragm 248. Sensor array 230 may include other sensors, not shown, for purposes of simplification. In this embodiment, diaphragm 248 has uneven outer surface 250 due to sensor 242 being larger than sensor 240 and sensor 244.

FIG. 2C is a cross section of a portion of sensor array 260, which includes sensor 270, sensor 272, and sensor 274 mounted between substrate 276 and diaphragm 278. Sensor array 260 may include other sensors, not shown, for purposes of simplification. In this embodiment, diaphragm 278 has uneven outer surface 280 due to the uneven surface of substrate 276, which causes the sensors to be disposed at non-uniform heights.

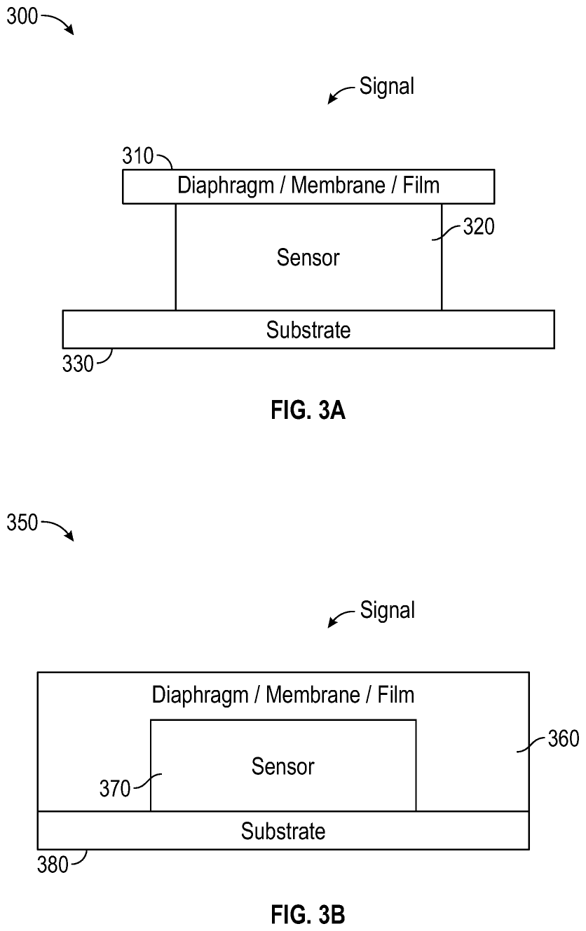

With respect to FIGS. 3A and 3B, different exemplary configurations of the diaphragm are described. FIG. 3A depicts a portion of sensor array 300 having diaphragm 310, sensor 320, and substrate 330. Likewise, FIG. 3B depicts a portion of sensor array 350 having diaphragm 360, sensor 370, and substrate 380. Diaphragm 310 is designed to be coupled to a single sensor, specifically sensor 320. In contrast, diaphragm 360 is designed to be coupled to two or more sensors, including sensor 370, and may be coupled to up to all of the sensors in the sensor array. It will be appreciated that in various embodiments, the diaphragm may engage a single surface of a membrane, as shown in FIG. 3A, whereas other embodiments may include a diaphragm that engages more than one surfaces of a sensor, as in FIG. 3B. In some embodiments, a sensor may be fully surrounded by the substrate and the diaphragm, while in other embodiments, the diaphragm or substrate may be omitted altogether.

In some embodiments, a sensor array may include one or more sensors, each having its own dedicated diaphragm that is not shared by any other sensor. In this case, the sensor array further may include a shared diaphragm that is coupled to a plurality of other sensors.

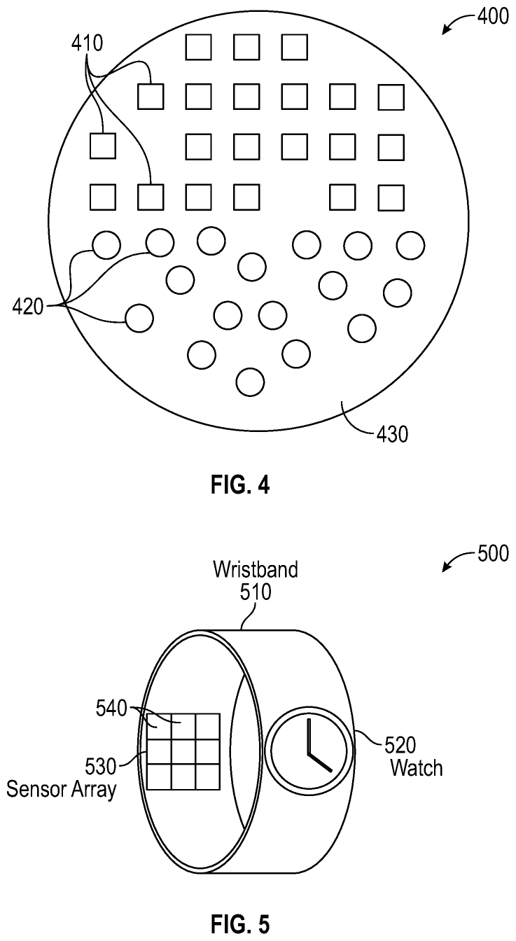

With respect to FIG. 4, another exemplary embodiment of a sensor array 400 constructed in accordance with the present invention is described. Sensor array includes a plurality of sensors 410 and a plurality of sensors 420 mounted to substrate 430, where sensors 410 are of a first type and sensors 420 are of a second type. Sensor arrays of the present invention may be configured to have any of a variety of shapes that may depend on the desired application, and sensor array 400 may be selected to have a circular shape to facilitate placement on the back side of a wristwatch having a circular body.

It will be appreciated that the sensors on a sensor array may have a number of configurations. In sensor array 400, sensors 410 generally are disposed in a grid-like configuration, whereas sensors 420 generally are disposed in a radial pattern. Notably, not all of the positions in either the grid-like or radial configurations must contain a sensor. While sensor array 400 includes sensors 410 in a first discrete area and sensors 420 in a second discrete area, it will be appreciated that other embodiments may include a plurality of different types of sensors in a discrete area.

In accordance with one aspect of the invention, use of a sensor array eliminates the need for precise placement of a single sensor. The lack of precision placement is advantageous in a clinical/ICU setting, in which the clinicians may be working under time constraints and do not have the benefit of extra time to properly place a sensor to obtain a preferred signal quality. Moreover, devices like tonometers frequently require repositioning and handling of the sensor during signal acquisition, thus distracting the clinician from other time-critical activities. Such forthcomings of previously known systems may be avoided using a device with a sensor array constructed in accordance with the present invention. Similarly, in a home-usage setting, lay users of the sensor, who may lack the necessary skill and training to properly position a device with a single sensing element may benefit from using a device with a sensor array to provide more accurate health monitoring.

Another advantage of the sensor array is that it diminishes intra- and inter-user variability, repeatability and reproducibility. It has been observed that other techniques used in clinical practice, such as arterial tonometry, are not easily reproducible and have high inter-observer variability. A sensor array in accordance with aspects of the present invention advantageously can reduce this problem by avoiding the requirement for extended handling and repositioning following the initial placement.

It will be recognized that the use of a sensor array and the proper selection/acquisition algorithm, described in greater detail below, may increase the capabilities of each sensor and significantly reduce cost. The signal from multiple sensors may be used to increase the overall accuracy and the sampling frequency, allowing the use of low cost, mass-produced commercial sensors to achieve a degree of measurement accuracy that would otherwise require more expensive sensing equipment. As another advantage, when a sensor array as described herein employs multiple different sensors, specific methodologies may be used to further improve the captured signal, such as stopping the acquisition when motion or muscle contraction is detected.

Alternative methods may be used to estimate the same cardiac index, and performing comparisons between methods can identify a desired method and yield more accurate results. In this regard, a comparison of different methodologies may identify outlying data for exclusion and may allow for correcting measurement differences between individuals.

Yet another embodiment of a sensor array configuration of the present invention is described with respect to FIG. 5. In FIG. 5, wearable device 500 is configured to be worn on a user's arm, and includes wristband 510 and watch 520. Wristband 510 preferably is adjustable so that it can be worn by people of having different wrist diameters. Wristband 510 may be self-adjusting, such as an elastic band that may stretch or contract to conform to different size wrists. Sensor array 530 includes a plurality of sensors 540 coupled to wristband 510 and sensor array 530 configured to be disposed adjacent to a user's body when wearable device 500 is worn by the user. In some embodiments, wristband 520 also may serve as the diaphragm of sensor array 530. Preferably, sensor array 530 includes a flexible substrate to conform with the movement of wristband 510.

Wearable device 500 may further include a second sensor array (not shown) disposed on the reverse side of watch 520. In some embodiments, the second sensor array may have a rigid substrate that is integral with the body of watch 520.

One of skill in the art will appreciate that sensor arrays constructed in accordance with the present invention may be configured in a wide variety of shapes and sizes, and further may include a plurality of different sensor types.

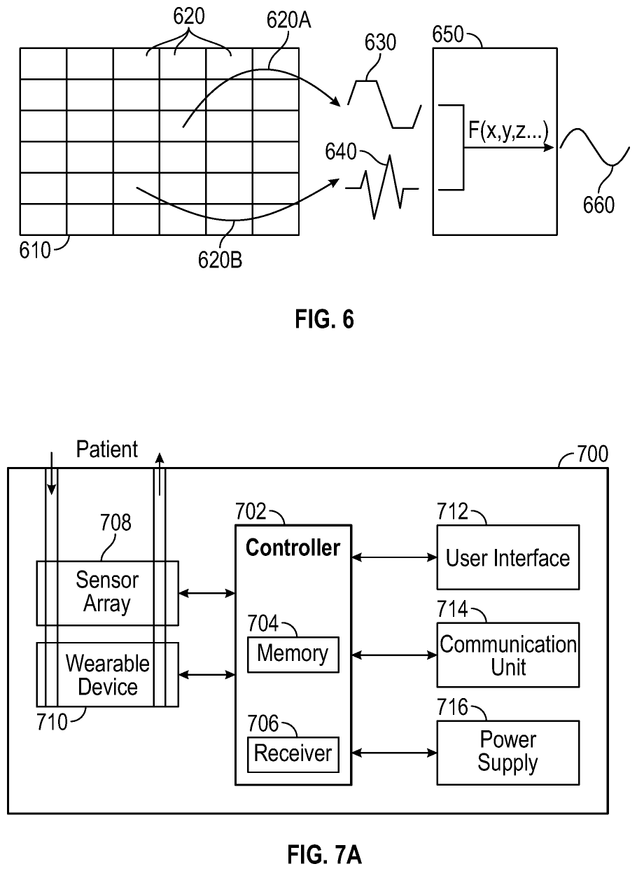

Data selection and acquisition is described with reference to FIG. 6. In the embodiment of FIG. 6, sensor array 610 has a plurality of sensors 620 that detect pulsation data and a pulsatile waveform of an arterial blood flow. Each of the plurality of sensors 620 transmits signals representative of the pulsation data and pulsatile waveform of the arterial blood flow. Sensor 620A transmits signal 630 and sensor 620B transmits signal 640. Signal 630 and signal 640 may be transmitted using wires or wirelessly. Though sensor 620A and sensor 620B may each send signals representative of pulsation data and a pulsatile waveform of the arterial blood flow, it will be appreciated that signal 630 and signal 640 may not be identical. For example, even if sensor 620A and sensor 620B detect pulsation data and a pulsation waveform of an arterial blood flow simultaneously, signal 630 may be different than signal 640 because sensor 620A and sensor 620B are located at slightly different positions relative to the patient's body. Additionally, sensor 620A and sensor 620B may be configured to acquire data at different times, such as sequentially. In this regard, sensor 620A may be configured to transmit data corresponding to a first heartbeat and sensor 620B may be configured to transmit data corresponding to a second heartbeat.

Receiver 650 is configured to receive and compare signals from sensor array 610, including signal 630 and signal 640. Receiver 650 may analyze a quality of signal 630 and signal 640. For example, receiver 650 may compare the signal quality to determine which of signal 630 and signal 640 has a more desirable signal-to-noise ratio, which may be used to identify which sensor(s) data to analyze and which sensor(s) data to discard. It may be desirable to repeat this signal quality analysis and comparison periodically to account for changes in signal quality that may arise from, for example, repositioning of the sensor array on the patient. Repetition of the analysis and comparison steps may occur at fixed or varied intervals or may occur in response to sensed movement, such as may be measured by an accelerometer coupled to the sensor array.

Receiver 650 includes a data selection component configured to select one or more signals used to form output signal 660. Preferably, a signal having a more desirable signal-to-noise ratio is selected to form the output signal, and that signal may be used as the output signal with or without modification. In other embodiments, a signal from one sensor may be combined with one or more signals from one or more other sensors. Combining the signals from two or more sensors may produce a signal with increased spatial and temporal resolution and signal quality. Signals from some sensors can be used to improve overall signal quality and to reduce variability by a combination of known signal processing techniques such as filtering, motion artifact detection, noise reduction, and baseline elimination. Such an example may involve a system having a motion detector and configured such that the detection of motion will initiate a response in which signal acquisition is stopped or motion artifacts are removed.

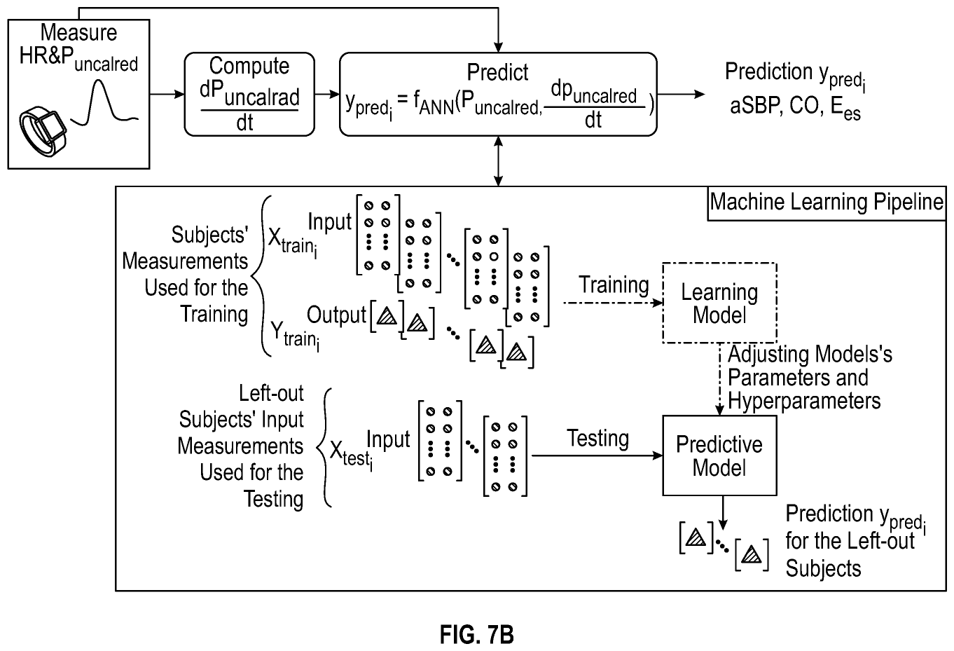

Referring now to FIG. 7A, a block diagram of an exemplary system constructed in accordance with the present invention is described. System 700 includes components that may be incorporated as an embodiment in which a controller is combined with a receiver. System 700 includes controller 702 having memory 704 and receiver 706. Controller may be a computer and memory may be a disk drive or flash memory. Controller 702 is in communication with sensor array 708 and wearable device 710. In some embodiments, sensor array 708 and wearable device 710 are integrated.

Controller 702 is also in communication with user interface 712, communication unit 714, and power supply 716. Controller 702 includes a processor that is programmed to perform an analysis on the signals received from sensor array 708, as described above. In some embodiments, wearable device 710 may, in response to communications from controller 702, display a representation of the analysis, such as by displaying an icon or text on a screen or illuminating a warning light. Wearable device 710 further may be configured to vibrate or issue an audible alert if an event generated by an analysis falls outside of a predetermined range. User interface 712 may include keyboard, mouse device, display screen, touch screen, or other user interface devices. Communication unit 714 may include alarm, WiFi, Internet, cloud storage, and telecommunication devices. Power supply 716 may include alternating current power or direct current power or may be switchable therebetween.

Controller 702 is configured to perform data analysis. In system 700, signals from sensor array 708 are transmitted to controller 702, where they are received by receiver 706. Controller 702 runs a the data analysis algorithm, which may run on a combination of CPU-RAM-ROM or a microcontroller.

Depending on the type of sensor that generated the signal, different types of signal processing algorithms, such as described in the above-incorporated PCT application may be applied such that the signal to be processed by an estimation algorithm, discussed below. The processing operations may include scaling, homogenization, calibration, recalibration, uncalibration, decreasing a sampling rate, or increasing a sampling rate. It has been observed that certain signals provide useful indices that can be extracted and transmitted to the estimation algorithm, such as a heart rate. Likewise, the inventors have observed that some types of signals benefit from additional processing to provide more useful information for the estimation algorithm. For example, a photoplethysmographic waveform may be transformed by an algorithm to a pressure waveform. Equivalent techniques may be applied to the other types of sensor outputs.

It will be understood that methods and/or algorithms of the present invention may be stored as programmed instructions, or as non-transitory computer-readable media, accessible to a processor of controller 702, thereby allowing programmed methods of the present invention, as described above, to be performed in a computer-controlled system.

With respect to FIG. 7B, a simplified estimation algorithm is described. The estimation algorithm uses as a sole input an uncalibrated radial blood pressure waveform, which may be acquired from a patient using a wrist-worn pressure sensor-array as described above. A raw uncalibrated pressure wave is provided to a machine learning-based system and an estimated value of interest, specifically aSBP, CO, and Eₑₛ, is provided. In the embodiment shown in FIG. 7B, a regression analysis was used in the training and testing the algorithms, and was performed using an artificial neural network (ANN). The estimation algorithm uses a model that incorporates data from a plurality of measurements, preferably taken from a plurality of test subjects. During training, the machine learning model associates certain traits or parameters with the desired output, which may be aSBP, CO, or Eₑₛ. Once trained, the machine learning model is presented with additional data to evaluate how closely the model predicts the output, which was not used during training phase. The model then may be adjusted in response to the test data to obtain increased accuracy in the predictive model. Other methods or deeper networks may also be utilized, such as a convolutional neural network.

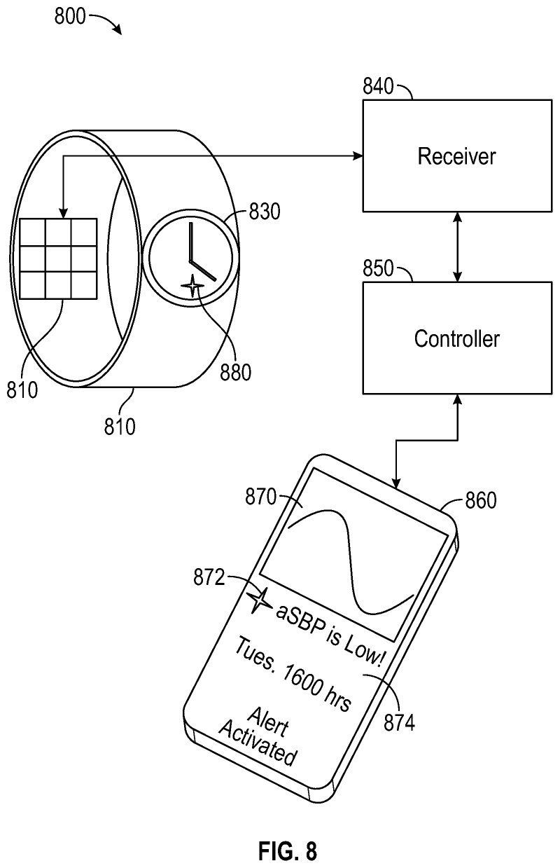

Referring now to FIG. 8, an illustrative embodiment of a system constructed in accordance with some aspects of the present invention is described. System 800 includes wearable device 810, which includes sensor array 820 and watch 830 mounted on a flexible substrate, which is configured to be worn on the wrist of a patient. Sensor array 820 includes a plurality of sensors mounted on the flexible substrate, wherein at least some of the sensors are configured to sense an arterial blood flow and to provide a signal representative of pulsation data and a pulsatile waveform for the arterial blood flow. The sensors may be mounted on one or more diaphragms as described above. Sensor array 820 is in communication with receiver 840, which receives the signals from the plurality of sensors in sensor array 820. Receiver 840 is configured to analyze a quality of each of the signals, such as a signal-to-noise ratio. Receiver 840 is further configured to determine an output signal based on a comparison of the qualities of the signals. This determination may in some embodiments involve a selection of a desired signal from a plurality of signals, and in other embodiments may involve generating a signal based on a combination of two or more signals. Receiver 840 is in communication with controller 850, which is configured to receive the output signal from receiver 840.

Controller 850 includes a processor and non-volatile storage for storing programming instructions. The programming instructions, when executed by controller 850, generate an estimate of at least one of aortic systolic blood pressure, cardiac output, or end-systolic elastance based on the output signal. In some embodiments, controller 850 utilizes a machine learning algorithm to generate the estimate. Controller 850 further may include a data recording component, such as a hard drive, flash drive, or other known data storage, which may store representations of the output signal or other information.

Controller 850 is in communication with device 860, which may be a phone, PDA, laptop computer, desktop computer, cloud storage, non-volatile storage, intranet, or other user interface device. Device 860 preferably includes display 870 configured to display a representation of the estimate of at least one of aortic systolic blood pressure, cardiac output, or end-systolic elastance. The display may take on many forms, such as a graphical representation, textual representation, or a warning light. Device 860 also may include an audio alert.

Device 860 may further be configured to provide a combination of outputs pertaining to the patient's cardiovascular health, including a visual representation of data on display screen 870, an alert icon 872, and textual data 874. Additionally, device 860 may be a communication device, such as a cell phone, that may be configured to communicate information pertaining to the patient's cardiovascular health to other recipients, such as a hospital, medical provider, or emergency response service. Device 860 may include a data recording component, such as described above, for storing information measured or generated by system 800.

In system 800, controller 850 further is configured to be in communication with wearable device 810. Controller 850 may communicate information pertaining to the patient's cardiovascular health to wearable device 810 which may result in an audio alert or announcement related to the communication. Likewise, wearable device may include display device 880 as part of watch 830, which may include one or more lights or a digital display screen.

The device depicted in FIG. 8, when employed with the proposed estimation algorithm, provides a scalable and versatile tool that reduces the required input measurements to only a single pulse wave. Specifically, it is expected that in clinical practice, machine learning-based technologies will present opportunities to improve the accessibility and performance of cardiovascular assessments over known techniques.

One advantage of machine learning technology used in accordance with the present invention is that a large amount of biomedical and clinical data is routinely collected that is suitable for training machine learning models to assess cardiovascular health. Advances in measurement techniques and systems have allowed for the acquisition of high-fidelity data suitable for assessing cardiovascular markers. Those large amounts of multi-dimensional and multi-variety data may be easily and efficiently handled by advanced machine learning algorithms.

Another advantage of machine learning technology used in accordance with the present invention stems from a rapid advancement in both hardware and software in recent years. The refinement of hardware components, such as high-performance processors and graphics processing units, has reduced the computational time required to train a machine learning model, even with large datasets, permitting predictions from the model to be made in real-time or near real-time.

Yet another advantage of machine learning technology used in accordance with the present invention related to the ability of machine learning systems to be automated such that they learn from data without human intercession. In particular, neural networks, adopted in the model discussed above, allowed for deciphering information hidden in the morphology of the pulse wave without the need for manual feature extraction.

Still another advantage of machine learning technology used in accordance with the present invention is that the machine learning-based algorithm provided easy, fast, and cost-efficient prediction of major biomarkers without the need for additional calibration, such as may be performed using a cuff-based pressure monitor. Avoiding the use of a cuff-based pressure monitor obviates the challenges stemming from their dependency on individual physiological parameters.

Accuracy and applicability of the disclosed invention has been demonstrated using a large in-silico cohort (n=3,818) that was generated using a previously validated cardiovascular model. The cohort was taken from “Determination of Aortic Characteristic Impedance and Total Arterial Compliance From Regional Pulse Wave Velocities Using Machine Learning: An in-silico Study,” by V. Bikia, G. Rovas, S. Pagoulatou, and N. Stergiopulos and published in May 2021 in Front. Bioeng. Biotechnol., vol. 9, p. 649866. The previously-validated model is available in “Validation of a one-dimensional model of the systemic arterial tree,” by Reymond, F. Merenda, F. Perren, D. Rüfenacht, and N. Stergiopulos and published in July 2009 in Am. J. Physiol. Heart Circ. Physiol., vol. 297, no. 1, pp. H208-222.

Predicted values were compared to reference data, and the results demonstrated that the estimators yielded precise predictions. Table 1 below provides the results of these predictions, where LoA is the limits of agreement within which 95% of errors are expected to lie.

TABLE 1

| | True | Estimated | | | |
|---|---|---|---|---|---|
| Marker | values | values | r | RMSE | Bias (LoA) |
| aSBP | 133.1 ± | 131.8 ± | 0.97 | 4.63 | −1.3 (−10.3, |
| [mmHg] | 17.6 | 16.5 | | | 7.7) |
| CO | 6 ± | 5.9 ± | 0.98 | 0.32 | −0.04 (−0.52, |
| [L/min] | 1.2 | 1.1 | | | 0.43) |
| Eₑₛ | 2.3 ± | 2.4 ± | 0.92 | 0.17 | 0.06 (−0.25, |
| [mmHg/mL] | 0.4 | 0.4 | | | 0.37) |

The results of Table 1 demonstrate that uncalibrated radial blood pressure wave may be highly informative for predicting the major cardiovascular markers, such as aSBP, CO, and Eₑₛ. Neural networks in accordance with this invention deciphered additional hidden information that allowed the derivation of those markers without the need for additional calibration. These findings demonstrate successful use of the disclosed machine learning algorithms to reveal more sophisticated aspects of vascular information through learning from the available data, leading to an active transformation of cardiovascular monitoring inside and outside the clinic.

It is to be understood that the implementations described herein are illustrative and that the scope of the present invention is not limited to those specific embodiments; many variations, modifications, additions, and improvements are possible. For example, functionality may be separated or combined in blocks differently in various embodiments of the disclosure or described with different terminology. These and other variations, modifications, additions, and improvements may fall within the scope of the disclosure as defined in the claims that follow.

What is claimed is:

1. A system for non-invasively monitoring cardiac health of a patient, the system comprising:
an array of sensors mounted to at least one diaphragm and configured to sense an arterial blood flow, a first sensor of the array providing a first signal, and a second sensor of the array providing a second signal;
a receiver configured to receive the first signal and the second signal, the receiver further configured to analyze a quality of each of the first signal and the second signal and to determine an output signal; and
a controller operatively coupled to the receiver, the controller having a processor and non-volatile storage for storing programmed instructions that, when executed by the controller, generates an estimate at least one of aortic systolic blood pressure, cardiac output, or end-systolic elastance based on the output signal.

2. The system of claim 1, wherein the controller generates the estimate employing a machine learning algorithm.

3. The system of claim 1, wherein the array of sensors is mounted on a flexible substrate.

4. The system of claim 1, wherein the receiver determines the output signal by comparing the quality of the first signal to the quality of the second signal to select one of the first signal and the second signal as the output signal.

5. The system of claim 1, wherein the receiver determines the output signal by combining the first signal and the second signal.

6. The system of claim 1, wherein the first sensor is selected from the group consisting of pressure sensors, force sensors, displacement sensors, acceleration sensors, photoplethysmographic sensors, light reflection sensors, and light sensors.

7. The system of claim 1, wherein at least a plurality of the sensors in the array of sensors are mounted on a common diaphragm.

8. The system of claim 1, wherein the receiver further is configured to receive the first signal at a different time than the second signal.

9. The system of claim 1, wherein the array of sensors is configured to circumscribe a portion of an arm of the patient.

10. The system of claim 1, wherein the array of sensors includes a motion sensor.

11. A system for non-invasively monitoring cardiac health of a patient, the system comprising:
a wearable device comprising:
a flexible substrate; and
an array of sensors coupled to the flexible substrate and configured to sense an arterial blood flow, a first sensor of the array providing a first signal, and a second sensor of the array providing a second signal;
a receiver configured to receive the first signal and the second signal, the receiver further configured to analyze a quality of each of the first signal and the second signal and to determine an output signal; and
a controller operatively coupled to the receiver, the controller having a processor and non-volatile storage for storing programmed instructions that, when executed by the controller, generates an estimate of at least one of aortic systolic blood pressure, cardiac output, or end-systolic elastance based on the output signal.

12. The system of claim 11, wherein the controller generates the estimate employing a machine learning algorithm.

13. The system of claim 11, wherein the receiver determines the output signal by comparing the quality of the first signal to the quality of the second signal to select one of the first signal and the second signal as the output signal.

14. The system of claim 11, wherein the receiver determines the output signal by combining the first signal and the second signal.

15. The system of claim 11, wherein the first sensor is selected from the group consisting of pressure sensors, force sensors, displacement sensors, acceleration sensors, photoplethysmographic sensors, light reflection sensors, and light sensors.

16. A system for non-invasively monitoring cardiac health of a patient, the system comprising:
a wearable device comprising:
a flexible substrate;
a first sensor mounted to the flexible substrate, the first sensor configured to sense an arterial blood flow and provide a first signal; and
a second sensor mounted to the flexible substrate, the second sensor configured to sense the arterial blood flow and provide a second signal;
a receiver configured to receive the first signal and the second signal, the receiver further configured to analyze a quality of each of the first signal and the second signal and to determine an output signal;
a controller operatively coupled to the receiver, the controller having a processor and non-volatile storage for storing programmed instructions that, when executed by the controller, generates an estimate of at least one of aortic systolic blood pressure, cardiac output, or end-systolic elastance based on the output signal; and
a display device in communication with the controller, the display device is configured to display a representation of the estimate.

17. The system of claim 16, wherein the controller generates the estimate employing a machine learning algorithm.

18. The system of claim 16, wherein the receiver determines the output signal by comparing the quality of the first signal to the quality of the second signal to select one of the first signal and the second signal as the output signal.

19. The system of claim 16, wherein the receiver determines the output signal by combining the first signal and the second signal.

20. The system of claim 16, wherein the first sensor is selected from the group consisting of pressure sensors, force sensors, displacement sensors, acceleration sensors, photoplethysmographic sensors, light reflection sensors, and light sensors.
